(19) United States
(12) Patent Application Publication
(10) Pub. No.: US 2026/0183406 A1
(43) Pub. Date: Jul. 2, 2026

(54) METHOD FOR PREPARING LINKER-DRUG CONJUGATE

(71) Applicant: LigaChem Biosciences Inc., Daejeon (KR)

(72) Inventors: Sei Chang Ahn, Daejeon (KR); Jeung Soon Choi, Daejeon (KR); Byung Soo Lee, Daejeon (KR); Jeong Soo Shim, Daejeon (KR)

(21) Appl. No.: 19/132,636

(22) PCT Filed: Nov. 21, 2023
(86) PCT No.: PCT/KR2023/018766

§ 371 Date: May 23, 2025

(30) Foreign Application Priority Data
Nov. 25, 2022 (KR) 10-2022-0160324

(51) Int. Cl. A61K 47/54; A61K 47/62

(52) U.S. Cl. CPC A61K 47/549; A61K 47/62

(57) ABSTRACT

The present disclosure relates to a method of preparing the linker-drug conjugate of Formula 1, wherein the manufacturing process is simple, safe, and has economic benefits.

### TECHNICAL FIELD

This application is based on and claims priority under 35 U.S.C. § 119 to Korean Patent Application No. 10-2022-0160324, filed on Nov. 25, 2022, in the Korean Intellectual Property Office, the disclosure of which is incorporated by reference herein in its entirety.

The present disclosure relates to a method of preparing a linker-drug conjugate.

### BACKGROUND ART

Antibodies are immunological proteins that bind to specific antigens, and numerous monoclonal antibodies are currently under development as anticancer agents or are used in the treatment of cancer. However, while nearly all antibodies can inhibit the proliferation of cancer cells and thereby impede cancer progression, their utility in treating cancer is quite limited. As an alternative approach to overcome these limitations, antibody-drug conjugates (ADCs), in which a drug is conjugated to an antibody, are being developed as a new form of antibody therapeutics.

An antibody-drug conjugate refers to the conjugation of a cytotoxic drug or toxin to a monoclonal antibody (mAb) capable of selectively delivering these agents or toxin to the interior of target tumor cells. When administered to a patient, the antibody-drug conjugate binds to target cells via its antibody portion and is delivered into the cell interior; subsequently, the cytotoxic drug or toxin is released from the antibody-drug conjugate and exhibits its own efficacy.

For the preparation of antibody-drug conjugates, the use of a suitable linker to effectively conjugate the antibody and the cytotoxic drug is standard practice. Representative examples of linkers used include hydrazone, disulfide, and peptide linkers. To function effectively, an antibody-drug conjugate requires that all three of its constituent components, namely the antibody, the linker, and the cytotoxic drug, exhibit functionality exceeding a certain threshold.

Antibody-drug conjugates incorporating the cytotoxic drug monomethyl auristatin E (MMAE) conjugated to an antibody are prepared through a multi-step process; a conventional method involves covalently linking the linker and MMAE in a liquid phase to form a linker-drug conjugate, followed by performing thiol or amino coupling with the antibody to form the antibody-drug conjugate. In conventional preparation methods, the toxic substance MMAE is conjugated to the linker at an early stage during the formation of the linker-drug conjugate, which necessitates significant caution and imposes constraints in handling the toxicity, thereby presenting economic challenges.

Therefore, a need exists for a method of preparing linker-drug conjugates that can resolve the aforementioned issues.

### DISCLOSURE OF INVENTION

### Technical Problem

As a result of conducting multifaceted research to solve the above issues, the present inventors have ascertained that, in a method of preparing the linker-drug conjugate of Formula 1 described herein, using bis(perfluorophenyl) carbonate as a reactant may reduce side reactions and improve reactivity, and introducing monomethyl auristatin E (MMAE), a drug with very high cytotoxicity, in the latter part of the manufacturing process may prevent long-term exposure to toxicity caused by MMAE and may simplify the manufacturing process, thereby completing the present disclosure.

Therefore, an object of the present disclosure is to provide a method of preparing the linker-drug conjugate of Formula 1 described herein.

### Solution to Problem

To achieve the aforementioned object,
the present disclosure provides a method of preparing a linker-drug conjugate of Formula 1, the method including: (1) obtaining a compound of Formula 4 by performing a condensation reaction between a compound of Formula 2 and a compound of Formula 3;
(2) obtaining a compound of Formula 5 by reacting the compound of Formula 4 and bis(perfluorophenyl) carbonate;
(3) obtaining a compound of Formula 6 by performing a condensation reaction between the compound of Formula 5 and monomethyl auristatin E (MMAE);
(4) obtaining a compound of Formula 7 by reacting the compound of Formula 6 and a base; and
(5) obtaining the compound of Formula 1 by reacting the compound of Formula 7
   and an acid.

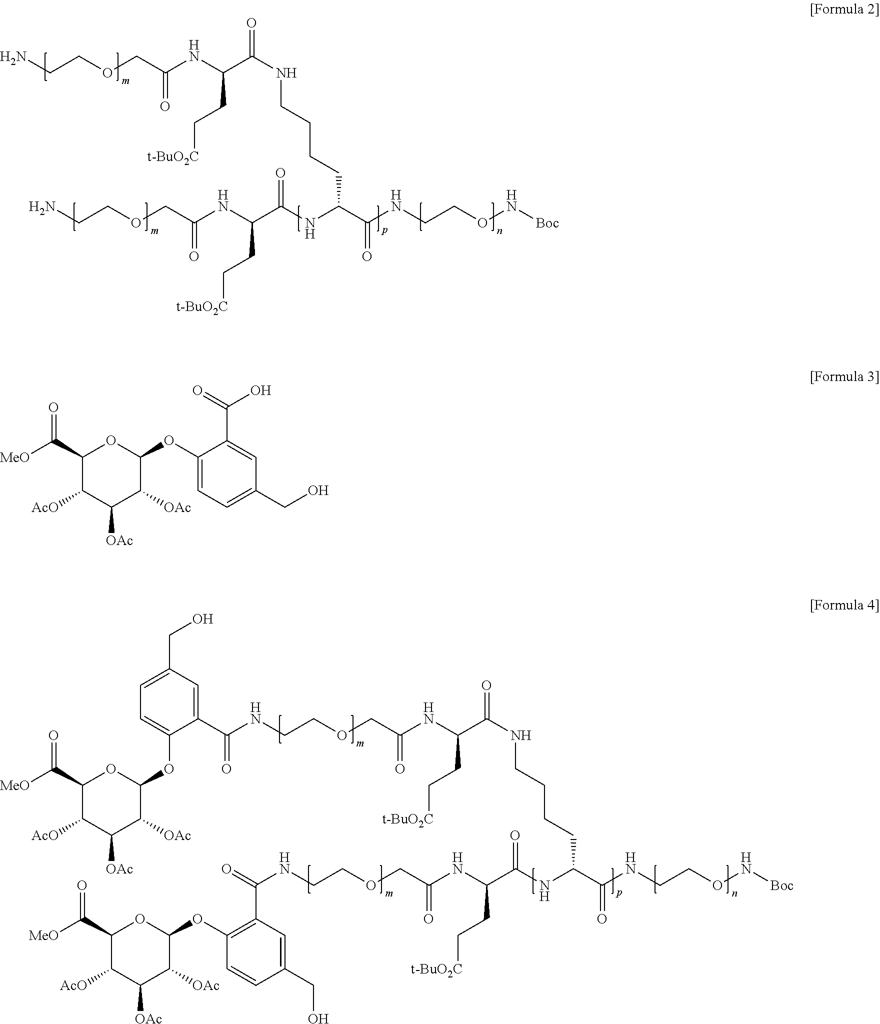

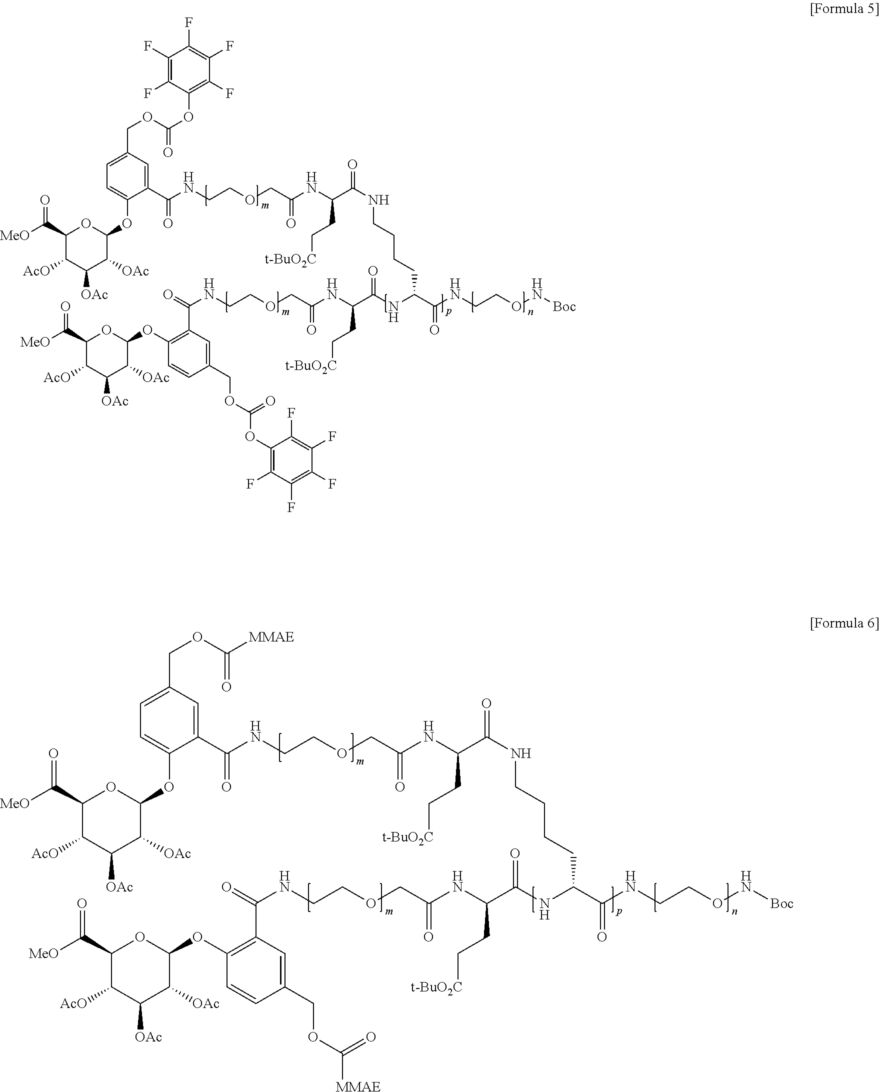

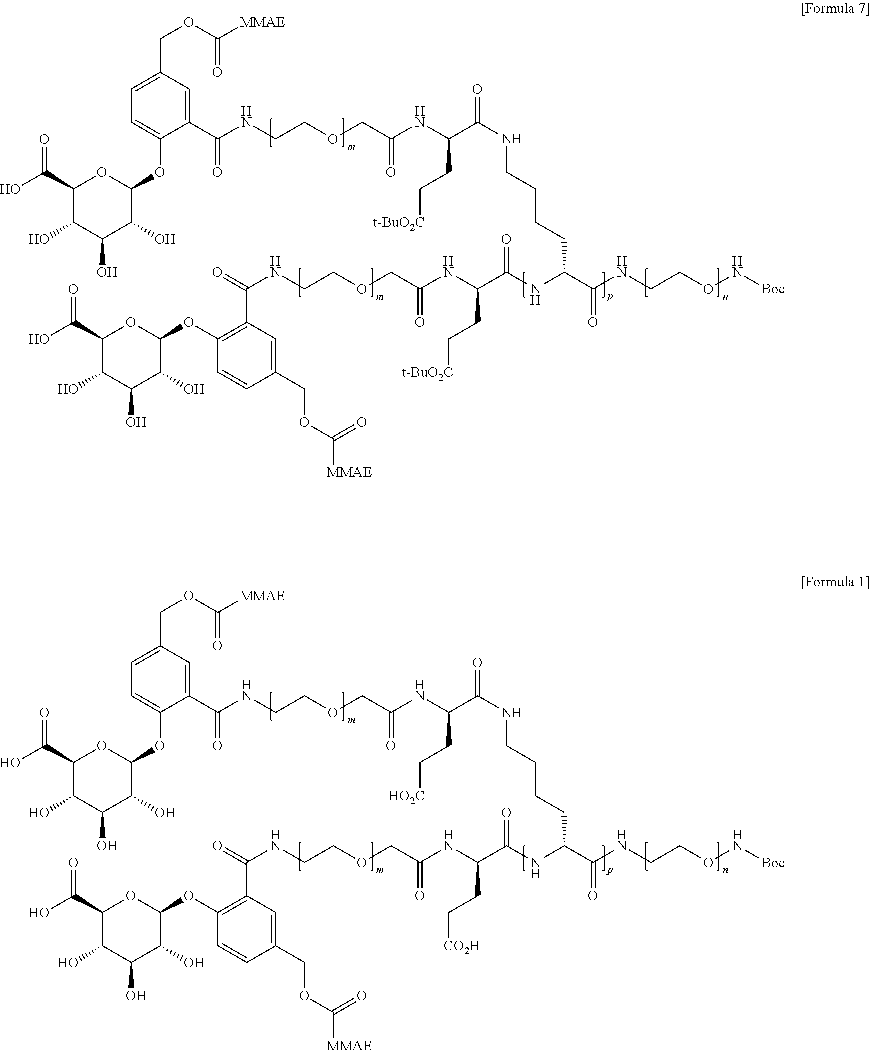

wherein, in Formulas 1 to 7,
p is an integer of 0 to 2,
m are the same or different from each other and are an integer of 1 to 6,
n is an integer of 1 to 6, and
MMAE is a compound of Formula 8.

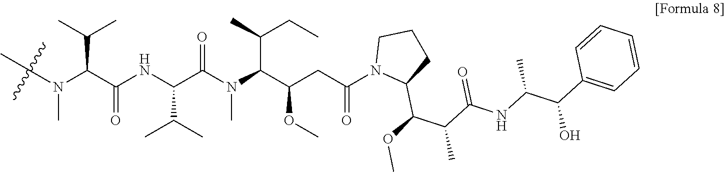 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

### Advantageous Effects of Invention

The method of preparing the linker-drug conjugate of Formula 1 according to the present disclosure involves a simple manufacturing process and can reduce the duration of exposure to toxicity, providing not only safety and economic benefits but also exhibiting excellent yield.

### BEST MODE FOR CARRYING OUT THE INVENTION

Hereinafter, the present disclosure will be described in greater detail.

A conventional method of preparing the linker-drug conjugate of Formula 1 is shown in Reaction Scheme 1.

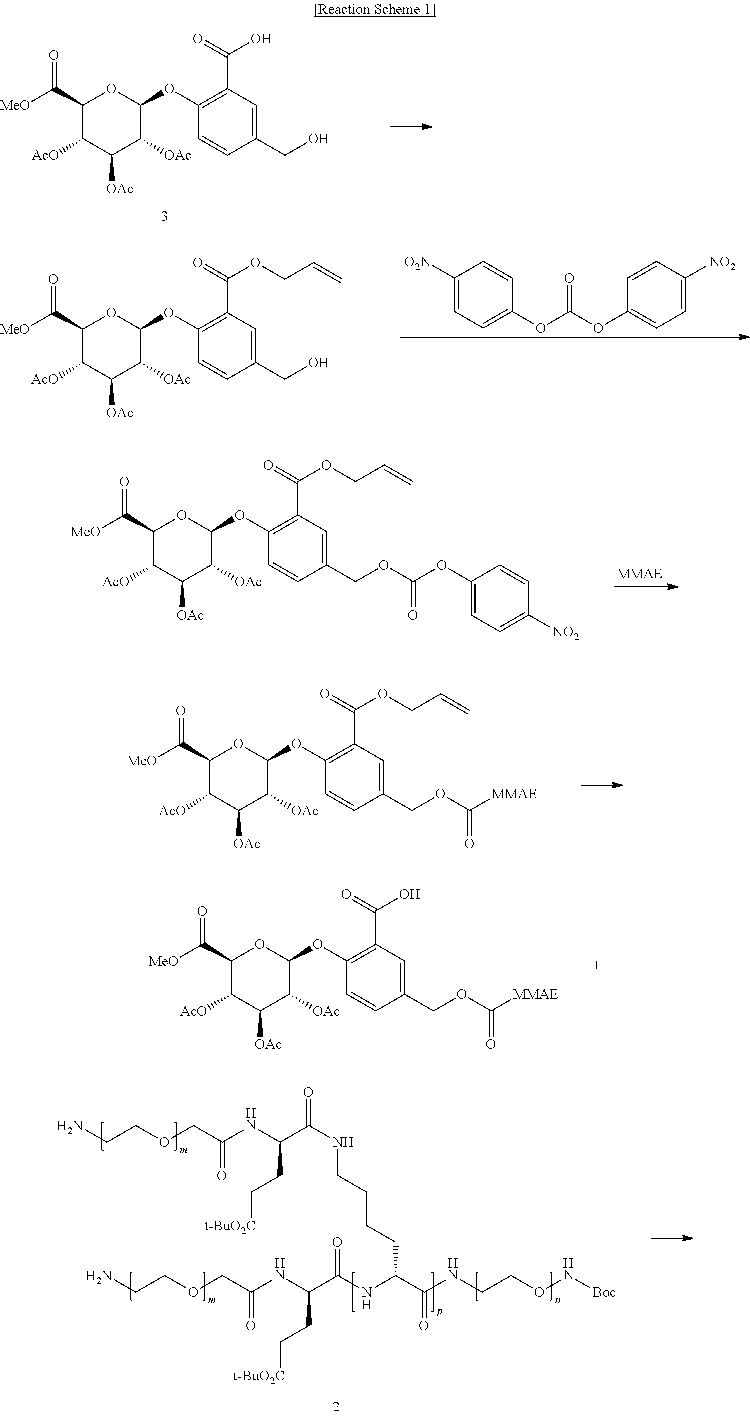

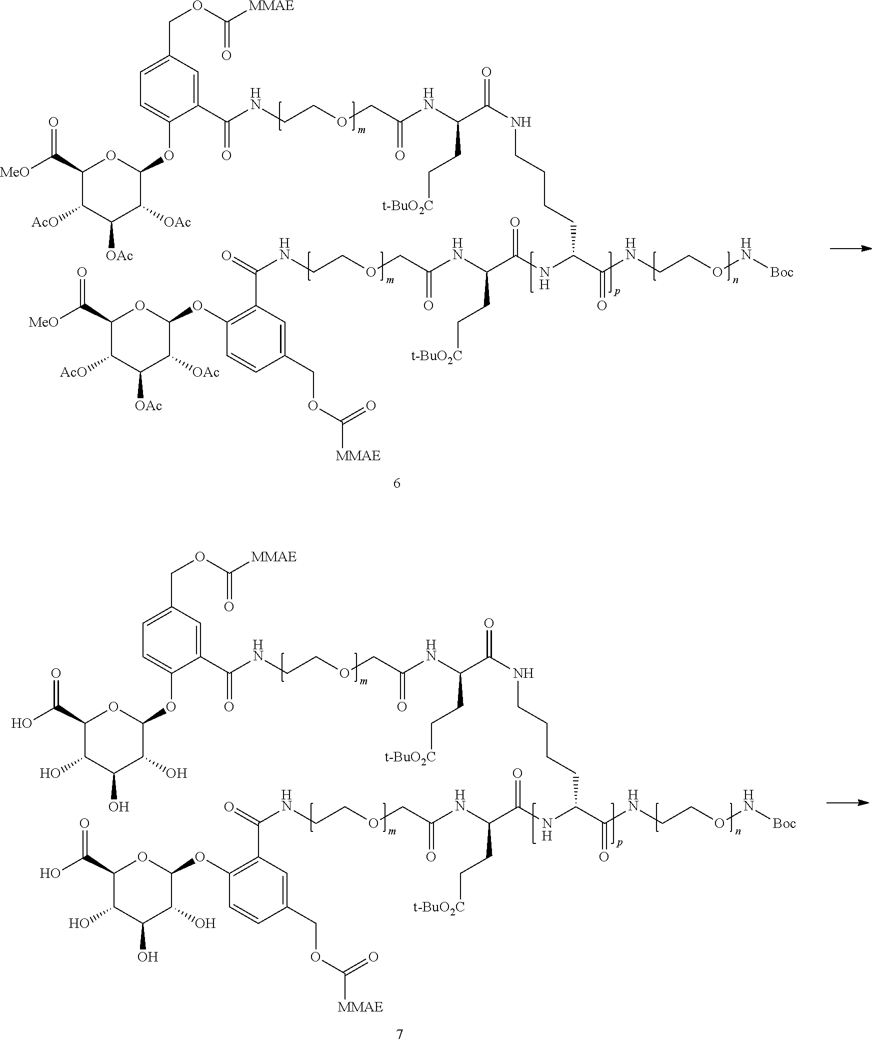

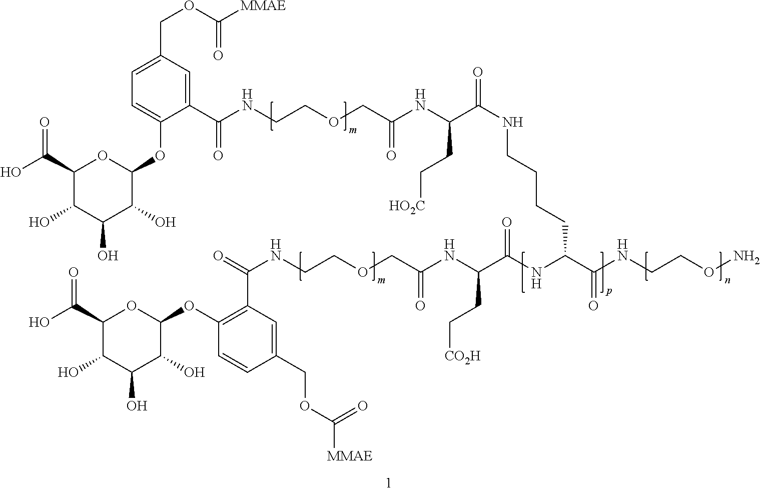

In the aforementioned conventional method of preparing the linker-drug conjugate of Formula 1, the cytotoxic substance monomethyl auristatin E (MMAE) is introduced and conjugated at the beginning of the manufacturing process; consequently, subsequent process steps involve continuous exposure to toxicity. Furthermore, preventing issues associated with this toxicity exposure requires significant caution and constraints during manufacturing, rendering the process uneconomical. Additionally, the manufacturing process is complex due to the requirement of performing numerous steps, and there is also the issue of very low yields, with the yield of the compound of Formula 6 being about 15% and the yield of the final product, the linker-drug conjugate of Formula 1, being about 15%.

The present disclosure seeks to provide a method of preparing the linker-drug conjugate of Formula 1 that can solve the aforementioned issues. In particular, the method of preparing the linker-drug conjugate of Formula 1 according to the present disclosure (a) allows for the reduction of toxicity exposure time by introducing and reacting the cytotoxic substance MMAE in the latter part of the manufacturing process. Furthermore, the method (b) reduces side reactions and improves reactivity when preparing the precursor for introducing monomethyl auristatin E (step (3) of the present disclosure) by using bis(perfluorophenyl) carbonate instead of bis(4-nitrophenyl) carbonate, and consequently, (c) the yield of the linker-drug conjugate of Formula 1 may be increased to about 60%. Furthermore, (d) the manufacturing process is simple and therefore economical.

The method of preparing the linker-drug conjugate of Formula 1 according to the present disclosure may include:
(1) obtaining a compound of Formula 4 by performing a condensation reaction between a compound of Formula 2 and a compound of Formula 3;
(2) obtaining a compound of Formula 5 by reacting the compound of Formula 4 and bis(perfluorophenyl) carbonate;
(3) obtaining a compound of Formula 6 by performing a condensation reaction between the compound of Formula 5 and MMAE;
(4) obtaining a compound of Formula 7 by reacting the compound of Formula 6 and a base; and
(5) obtaining a compound of Formula 1 by reacting the compound of Formula 7 and an acid.

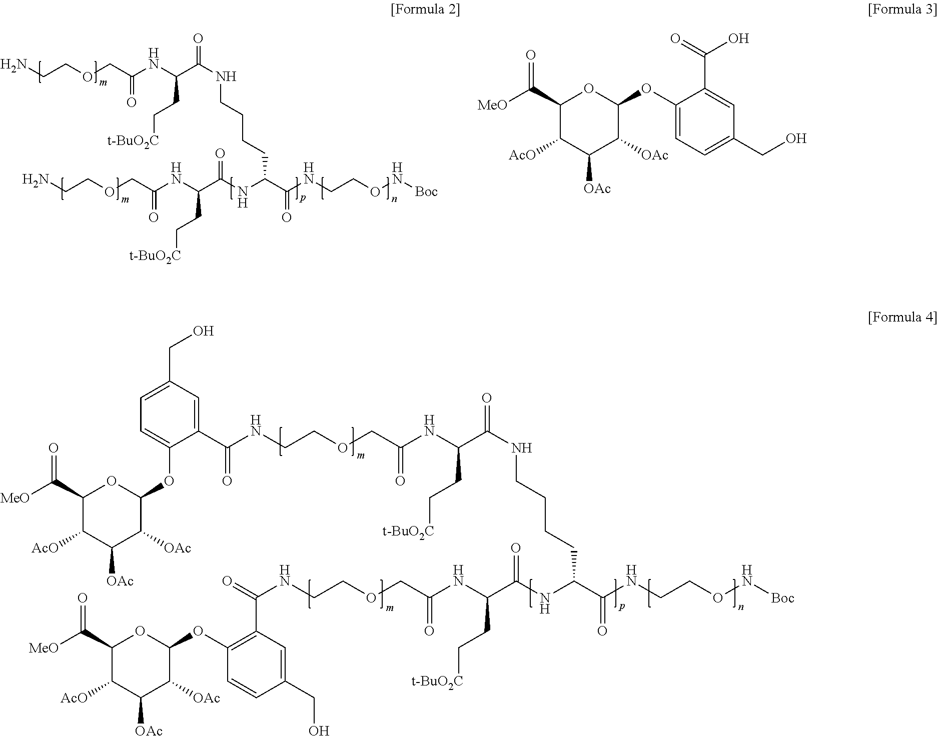

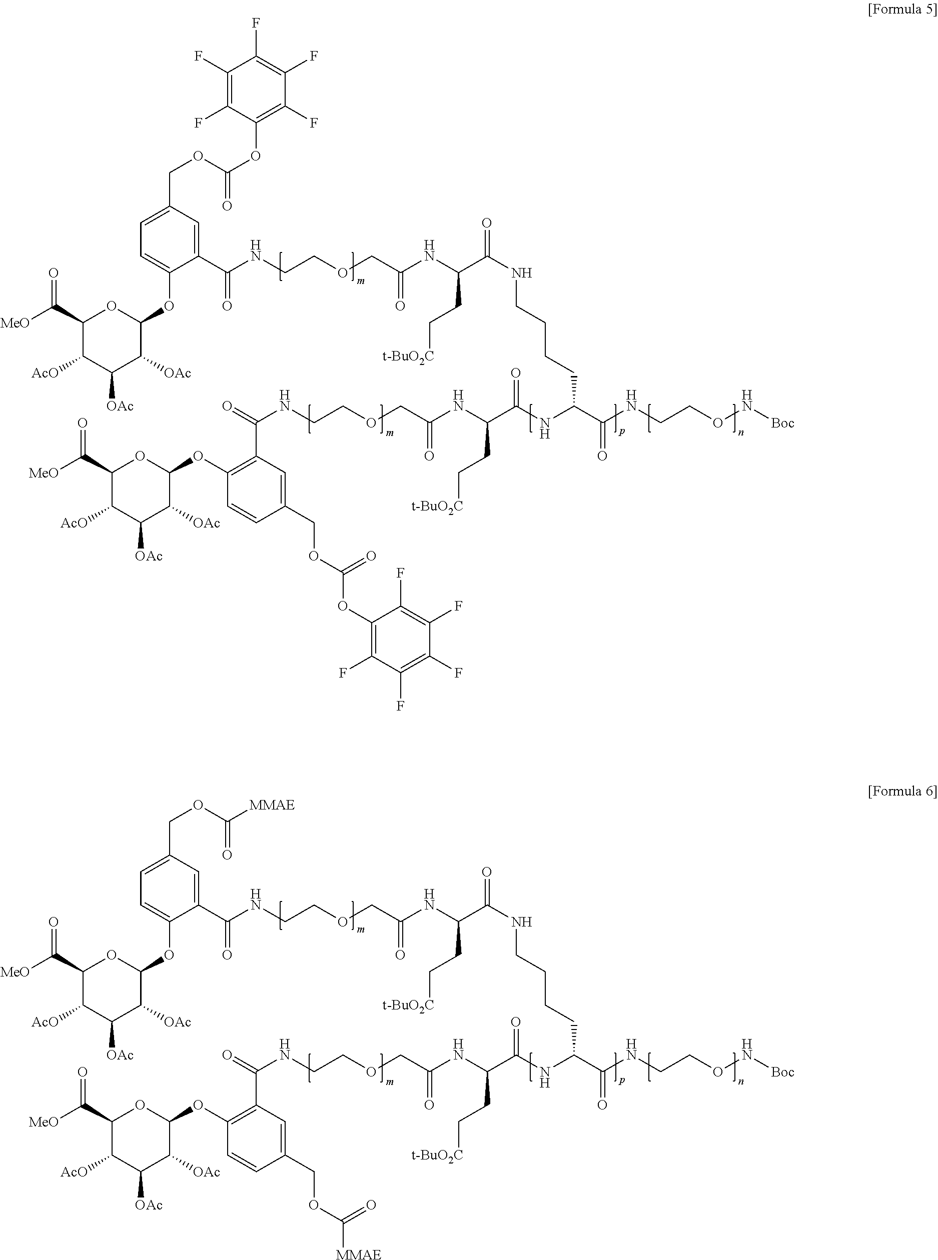

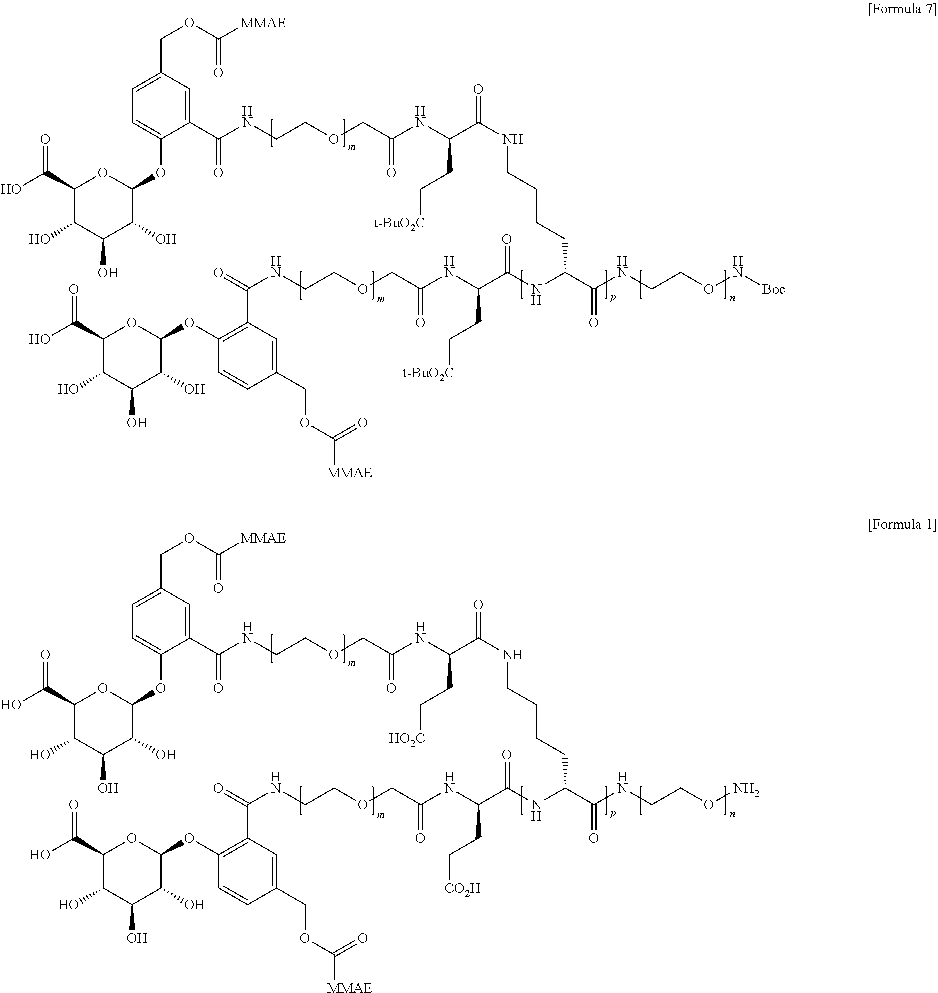

wherein, in Formulas 1 to 7,
p is an integer of 0 to 2,
m are the same or different from each other and are an integer of 1 to 6,
n is an integer of 1 to 6, and
MMAE is a compound of Formula 8.

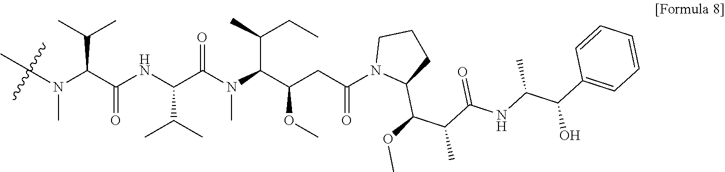 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

Step (1) is the step of obtaining the compound of Formula 4 by performing a condensation reaction between the compound of Formula 2 and the compound of Formula 3.

The condensation reaction of step (1) may be performed in the presence of a condensation agent including one or more selected from the group consisting of carboxylic acid activators, racemization inhibitors, and bases.

Specifically, the carboxylic acid activator may include one or more selected from the group consisting of carbodiimides, uronium salts, phosphonium salts, and phosphonic anhydrides.

The carbodiimides may preferably include one or more selected from the group consisting of 1-ethyl-3-(3-dimethylaminopropyl) carbodiimide hydrochloride (EDC·HCl), dicyclohexylcarbodiimide (DCC), and diisopropylcarbodiimide (DIC).

The uronium salts may preferably include one or more selected from the group consisting of (benzotriazol-1-yloxy)tris(dimethylamino)phosphonium hexafluorophosphate (BOP), 2-(7-aza-1H-benzotriazol-1-yl)-1,1,3,3-tetramethyluronium hexafluorophosphate, N,N,N′,N′-tetramethyl-O-(7-azabenzotriazol-1-yl) uronium hexafluorophosphate (HATU), and O-(benzotriazol-1-yl)-N,N,N′,N′-tetramethyluronium hexafluorophosphate (HBTU).

The phosphonium salts may preferably include benzotriazol-1-yl-oxytripyrrolidinophosphonium hexafluorophosphate (PyBOP).

The phosphonic anhydrides may preferably include propylphosphonic anhydride (T3P).

The racemization inhibitor may preferably include one or more selected from the group consisting of hydroxybenzotriazole (HOBt) and 1-hydroxy-7-azabenzotriazole (HOAt).

The base may preferably include one or more selected from the group consisting of triethylamine (TEA), N,N-diisopropylethylamine (DIPEA), and N-methylmorpholine (NMM).

The condensation reaction may be performed in the presence of preferably one or more selected from the group consisting of 1-ethyl-3-(3-dimethylaminopropyl) carbodiimide hydrochloride, hydroxybenzotriazole, and N-methylmorpholine, and most preferably may be performed in the presence of 1-ethyl-3-(3-dimethylaminopropyl) carbodiimide hydrochloride, hydroxybenzotriazole, and N-methylmorpholine. In an embodiment, the compound of Formula 3, 1-ethyl-3-(3-dimethylaminopropyl) carbodiimide hydrochloride, hydroxybenzotriazole, and N-methylmorpholine may be added in identical equivalents.

Step (1) may include: (1-1) mixing the compound of Formula 2 and the compound of Formula 3, followed by cooling to −10 to 5° C.;
(1-2) adding the condensation agent at the aforementioned temperature; and
(1-3) stirring the mixture at room temperature.

Step (1-1) is mixing the compound of Formula 2 and the compound of Formula 3 in an organic solvent, wherein the organic solvent may include one or more selected from the group consisting of N,N-dimethylformamide (DMF), dichloromethane, and tetrahydrofuran (THF). Preferably, the organic solvent may include dichloromethane.

The compound of Formula 2 and the compound of Formula 3 may be mixed in an equivalent ratio of 1:1.5 to 1:3, and preferably may be mixed in an equivalent ratio of 1:1.8 to 1:2.5.

Furthermore, the cooling temperature may be −10 to 5° C., and preferably may be −5 to 5° C.

Step (1-3) is stirring the mixture prepared in step (1-2) at room temperature, which may be performed under a nitrogen (N₂) atmosphere, and the compound of Formula 4 may be prepared in this step. Subsequently, washing and purification may be additionally performed, wherein the purification method is not particularly limited, provided that it is used in the relevant technical field, and for example, purification may be carried out by chromatography.

Step (2) is obtaining the compound of Formula 5 by reacting the compound of Formula 4 and bis(perfluorophenyl) carbonate.

Bis(perfluorophenyl) carbonate exhibits superior reactivity towards nucleophiles compared to bis(4-nitrophenyl) carbonate, thereby enhancing the nucleophilic reaction when introducing monomethyl auristatin E (MMAE) in step (3) described later.

Therefore, side reactions, such as the conjugation of only one MMAE molecule, may be reduced, and reactivity may be improved, making it possible to increase the yield of the linker-drug conjugate of Formula 1.

Step (2) may be performed in the presence of an organic base, wherein the organic base may include one or more selected from the group consisting of pyridine, triethylamine, and N-methylmorpholine, and may preferably include pyridine. In an embodiment, the bis(perfluorophenyl) carbonate and the organic base may be used in identical equivalents.

Furthermore, step (2) may be performed by dissolving the compound of Formula 4 in an organic solvent, followed by adding the organic base and bis(perfluorophenyl) carbonate. The organic solvent may include one or more selected from the group consisting of N,N-dimethylformamide (DMF), dichloromethane, and tetrahydrofuran (THF), and may preferably include dichloromethane.

Furthermore, step (2) may be performed at room temperature for 10 to 25 hours. Subsequently, washing may be additionally performed, and purification may be performed as needed. The purification method is not particularly limited provided that it is used in the relevant technical field, and for example, purification may be carried out by chromatography.

Step (3) is obtaining the compound of Formula 6 by performing a condensation reaction between the compound of Formula 5 and monomethyl auristatin E (MMAE).

MMAE may be the compound of Formula 8.

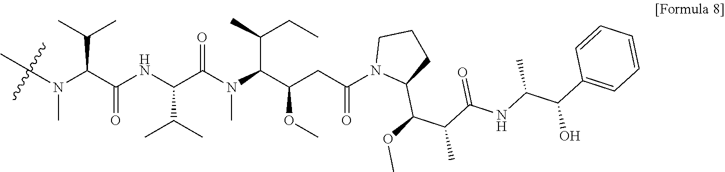 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

Therefore, the compound of Formula 6 may be represented by the structure of Formula 6-1.

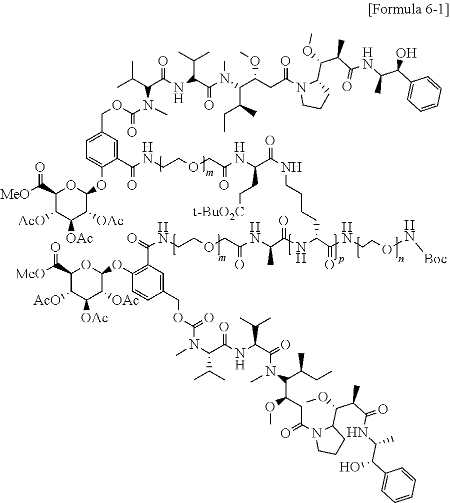

Herein, p, m, and n are as defined in Formula 6.

MMAE is a cytotoxic substance, possessing strong toxicity. As described above, conventionally, in the preparation of the linker-drug conjugate of Formula 1, MMAE is introduced early, leading to the issue of continuous exposure to toxicity in subsequent process steps. However, the present disclosure may solve the aforementioned issues by introducing MMAE in the latter part of the manufacturing process.

MMAE may be added in an amount of 2 to 3 equivalents.

The condensation reaction of step (3) may be performed in the presence of a condensation agent and an organic base. The condensation agent may include one or more selected from the group consisting of hydroxyazabenzotriazole (HOAt) and hydroxybenzotriazole (HOBt), and may preferably include hydroxyazabenzotriazole. The organic base may include one or more selected from the group consisting of diisopropylethylamine (DIEA), triethylamine, and pyridine, and may preferably include diisopropylethylamine. In an embodiment, the condensation agent and the organic base may be used in an equivalent ratio of 1:1 to 1:5, and most preferably may be used in an equivalent ratio of 1:5.

Furthermore, step (3) may be performed by dissolving the compound of Formula 5 in an organic solvent, followed by adding the condensation agent, the organic base, and MMAE. The organic solvent may include one or more selected from the group consisting of N,N-dimethylformamide (DMF), dichloromethane, and tetrahydrofuran (THF), and may preferably include N,N-dimethylformamide.

Furthermore, step (3) may be adding the compound of Formula 5, MMAE, the organic base, and the condensation agent at a temperature of −10 to 0° C., and reacting at a temperature of 0 to 25° C. for 10 to 25 hours.

Subsequently, washing may be additionally performed, and purification may be performed as needed. The purification method is not particularly limited provided that it is used in the relevant technical field, and for example, purification may be carried out by chromatography.

The yield of the compound of Formula 6 is about 60%, which is markedly higher than the 15% yield of the compound of Formula 6 prepared by the aforementioned conventional method of preparing the linker-drug conjugate of Formula 1, representing an improvement in yield of about four times or more.

Step (4) may be obtaining the compound of Formula 7 by reacting the compound of Formula 6 and a base; this reaction may be performed at a low temperature.

The base may include one or more selected from the group consisting of lithium hydroxide, sodium hydroxide, and calcium hydroxide, preferably may include lithium hydroxide, and the lithium hydroxide may be lithium hydroxide monohydrate.

Specifically, step (4) may be dissolving the compound of Formula 6 in an organic solvent, adding the base dropwise thereto at −50 to −20° C., and then carrying out the reaction under a nitrogen (N₂) atmosphere at −20 to 5° C. for 2 to 5 hours. The organic solvent may include one or more selected from the group consisting of methanol, N,N-dimethylformamide (DMF), dichloromethane, and tetrahydrofuran (THF), and may preferably include methanol and tetrahydrofuran. The base may be an aqueous base solution. Subsequently, prior to performing step (5), neutralization of the compound of Formula 7 to pH 6 to 8 may be additionally performed, wherein the neutralization may be carried out using a weak acid such as acetic acid or trifluoroacetic acid.

Furthermore, since MMAE is the compound of Formula 8, the compound of Formula 7 may be represented by the structure of Formula 7-1.

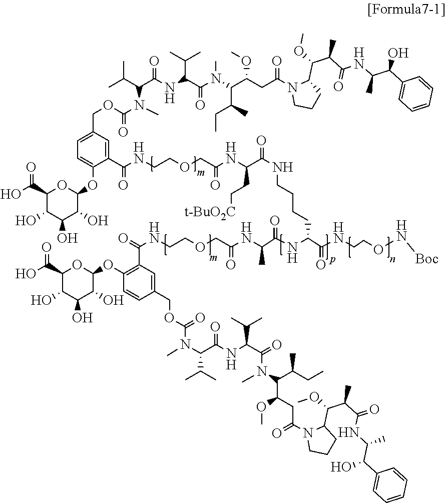

Herein, p, m, and n are as defined in Formula 7.

Step (5) may be obtaining the linker-drug conjugate of Formula 1 by reacting the compound of Formula 7 and an acid, wherein the acid may include one or more selected from the group consisting of phosphoric acid, trifluoroacetic acid, sulfuric acid, and acetic acid, and may preferably include phosphoric acid.

Step (5) may be dissolving the compound of Formula 7 in an organic solvent, adding the acid dropwise thereto at −10 to 10° C., and then carrying out the reaction at −10 to 10° C. for 1 to 5 hours. The organic solvent may include one or more selected from the group consisting of N,N-dimethylformamide (DMF), dichloromethane, acetonitrile, and tetrahydrofuran (THF), and may preferably include dichloromethane.

Subsequently, purification may be additionally performed. The purification method is not particularly limited, provided that it is used in the relevant technical field, and for example, prep-HPLC purification may be carried out. After the purification, the final linker-drug conjugate of Formula 1 was obtained by freeze-drying, and the yield may be about 25%.

Furthermore, since MMAE is the compound of Formula 8, the linker-drug conjugate of Formula 1 may be represented by the structure of Formula 1-1.

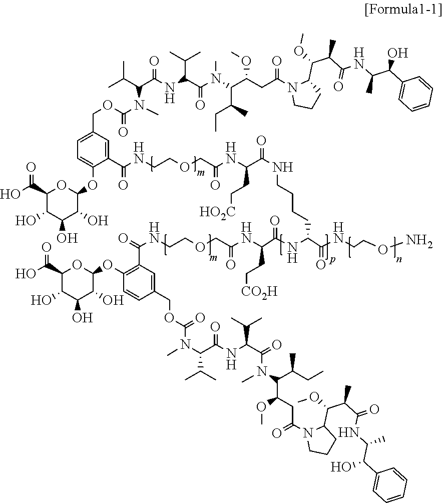

Herein, p, m, and n are as defined in Formula 1.

Furthermore, when p is 0, the linker-drug conjugate of Formula 1 may be the linker-drug conjugate of Formula 1-2.

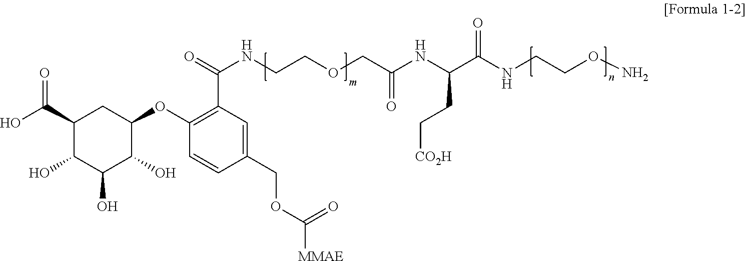

Herein, m and n are as defined in Formula 1.

Furthermore, when p is 1, the linker-drug conjugate of Formula 1 may be the linker-drug conjugate of Formula 1-3.

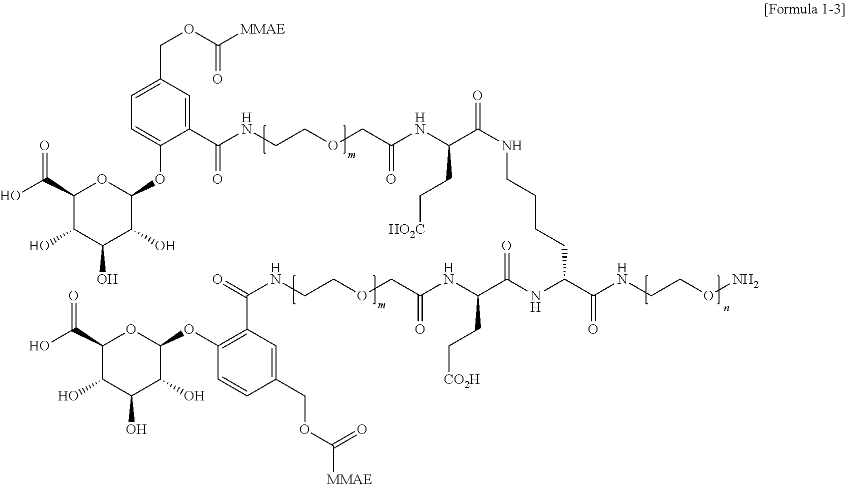

Herein, m and n are as defined in Formula 1.

Furthermore, when p is 2, the linker-drug conjugate of Formula 1 may be the linker-drug conjugate of Formula 1-4.

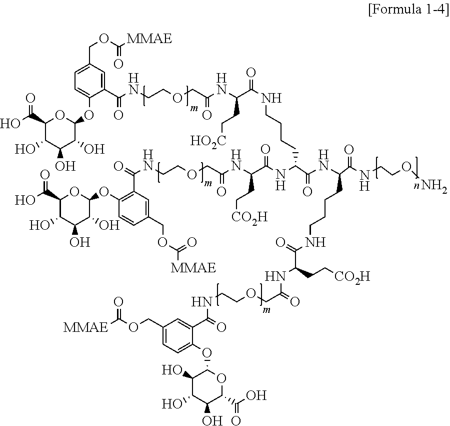

Herein, m and n are as defined in Formula 1.

That is, according to the definition of p, the number of conjugated MMAE moieties in the linker-drug conjugate of Formula 1 varies and may range from 1 to 3.

The method of preparing the linker-drug conjugate of Formula 1 according to the present disclosure may be represented by Reaction Scheme 2.

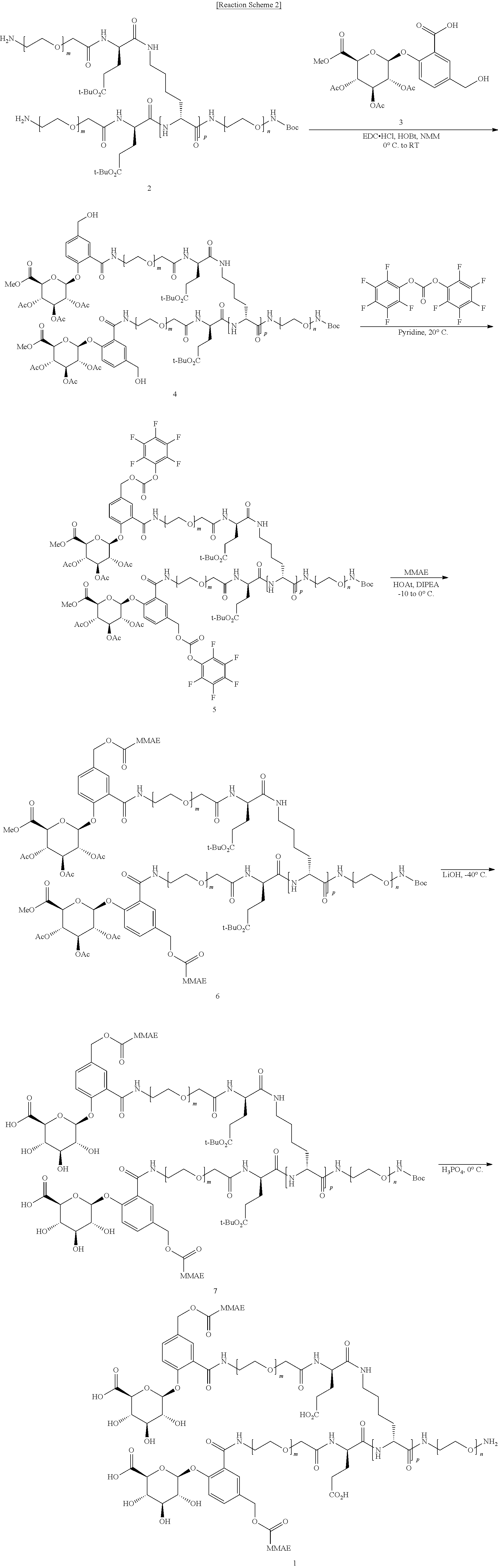

### MODE FOR THE INVENTION

Hereinafter, preferred examples are presented to aid understanding of the present disclosure, but the following examples are merely illustrative of the present disclosure. It is apparent to those skilled in the art that various changes and modifications may be made within the scope and spirit of the present disclosure, and such variations and modifications also fall within the scope of the appended claims.

### <Preparation of Linker-Drug Conjugate of Formula 1>

### Example 1

### Example 1-1. Preparation of Compound of Formula 4

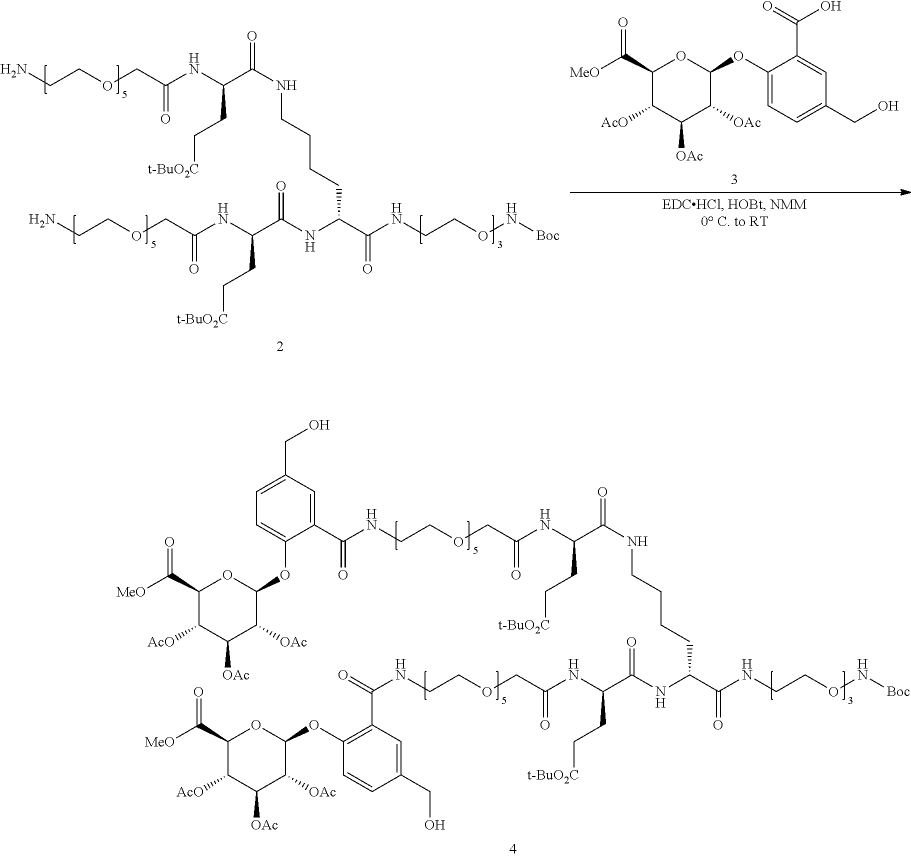

The compound of Formula 2 (5.31 g, 4.03 mmol), the compound of Formula 3 (3.55 g, 7.33 mmol, 1.82 eq), and dichloromethane (CH₂Cl₂, DCM) (27 mL, 5 V) were combined and cooled to 0° C.

To the reaction mixture, a solution of 1-ethyl-3-(3-dimethylaminopropyl) carbodiimide hydrochloride (EDC·HCl) (1.41 g, 7.33 mmol, 1.82 eq), hydroxybenzotriazole (HOBt) (0.99 g, 7.33 mmol, 1.82 eq), and N-methylmorpholine (NMM) (0.81 mL, 7.33 mmol, 1.82 eq) dissolved in dichloromethane (CH₂Cl₂, DCM) (27 mL, 5 V) was added at 0° C.

The reaction mixture was stirred under a nitrogen atmosphere at a temperature of 20 to 25° C. until the reaction was complete.

Subsequently, the mixture was washed sequentially with 10% aqueous NH₄Cl solution (53 mL, 10 V), 10% aqueous NaHCO₃ solution (53 mL, 10 V), and water (53 mL, 10 V), dried over anhydrous sodium sulfate, and concentrated under reduced pressure.

The concentrate was purified by column chromatography (SiO₂, 2% methanol/dichloromethane→10% methanol/dichloromethane) to obtain the compound of Formula 4 (6.64 g, 81%) as a white solid.

The Mass and NMR results for the compound of Formula 4 are as follows.

EI-MS m/z: [M+H]⁺1318.6

¹H-NMR (400 MHz, CDCl₃), 0:7.77-7.76 (d, J=6.2 Hz, 2H), 7.50-7.47 (m, 2H), 7.25-7.23 (d, J=6.4 Hz, 2H), 5.63-5.61 (d, J=6.2 Hz, 2H), 5.52-5.49 (t, J=5.8 Hz, 2H), 5.36-5.34 (t, J=6.4 Hz, 2H), 5.24-5.22 (t, J=6.2 Hz, 2H), 4.60 (s, 4H), 4.56-4.53 (t, J=8 Hz, 2H), 4.45-4.35 (m, 2H), 4.33-4.30 (m, 1H), 4.05 (s, 4H), 3.73-3.71 (m, 2H), 3.70-3.64 (m, 56H), 3.40-3.30 (m, 2H), 3.21-3.19 (m, 2H), 2.39-2.31 (m, 4H), 2.11-2.05 (m, 20H), 1.96-1.85 (m, 2H), 1.85-1.76 (m, 1H), 1.69-1.60 (m, 1H), 1.54-1.38 (m, 31H)

### Example 1-2. Preparation of Compound of Formula 5

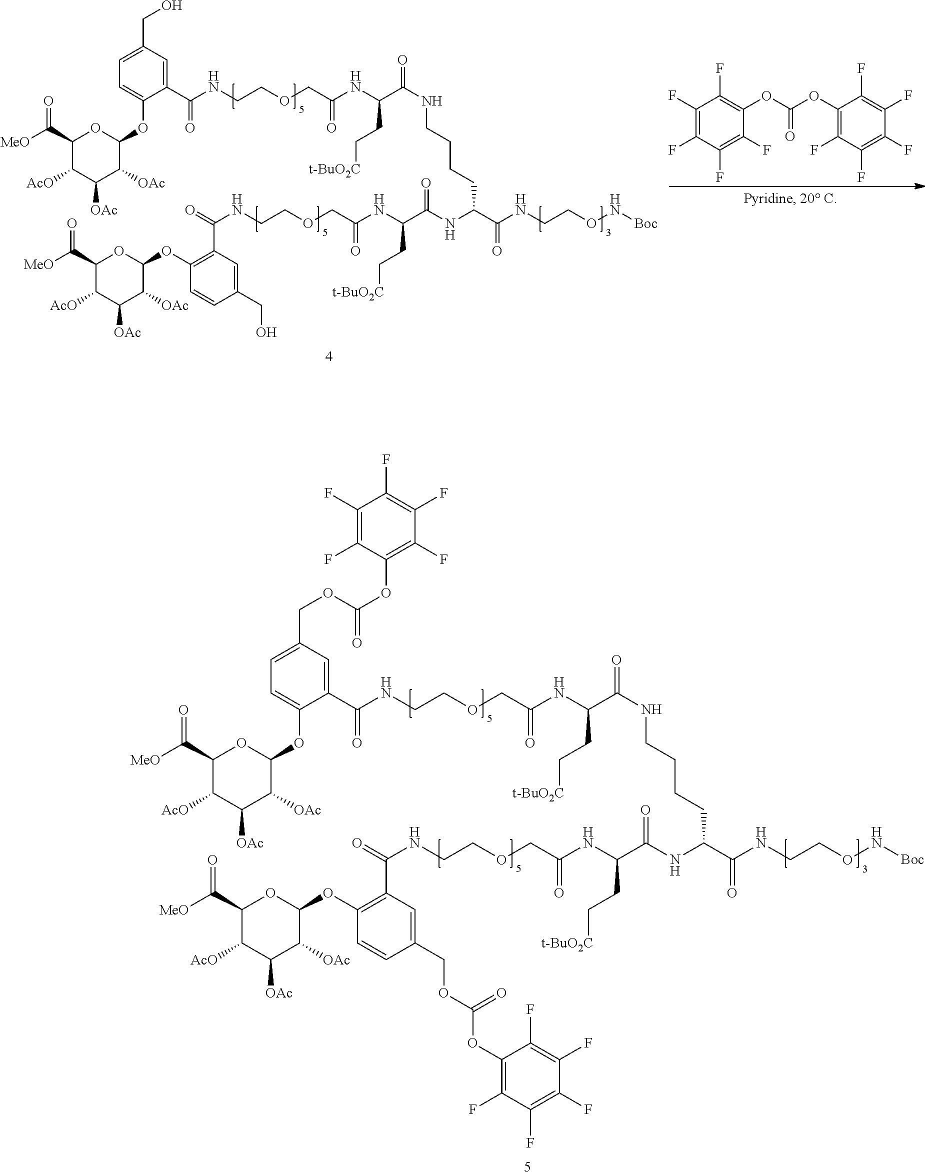

To a solution of the compound of Formula 4 (175 g, 73.9 mmol) dissolved in dichloromethane (CH₂Cl₂, DCM) (3.5 L, 20 V), pyridine (Py) (29.2 g, 370 mmol, 5.00 eq) and bis(perfluorophenyl) carbonate (146 g, 370 mmol, 5.00 eq) were sequentially added under a nitrogen atmosphere at a temperature of 20 to 25° C., followed by stirring for 12 hours until the reaction was complete.

The reaction mixture was washed with 10% aqueous NH₄Cl solution (3.50 L) and brine (3.50 L), dried over anhydrous sodium sulfate, and concentrated under reduced pressure.

The concentrate was purified by column chromatography (SiO₂, 100% ethyl acetate→dichloromethane:tetrahydrofuran (1/1, v/v)) to obtain the compound of Formula 5 (165 g, 78.6%).

The Mass and NMR results for the compound of Formula 5 are as follows.

EI-MS m/z: [½M+H]⁺1336.2, [⅓M+H]⁺891.0

¹H-NMR (400 MHz, CDCl₃), δ: 8.41 (br, 1H), 8.12 (br, 2H), 7.54˜7.49 (m, 4H), 7.42 (m, 2H) 7.40 (m, 1H), 7.12-7.09 (m, 3H), 7.03 (m, 1H), 5.44-5.33 (m, 7H), 4.48-4.46 (m, 3H), 4.26-4.24 (m, 2H), 4.04 (br, 6H), 3.76-3.56 (m, 57H), 3.45 (m, 2H), 3.25 (m, 1H), 3.15 (m, 1H), 2.36˜2.31 (m, 5H), 2.22 (br, 6H), 2.14 (m, 3H), 2.12 (s, 9H), 2.11 (s, 3H), 2.02 (m, 3H), 1.51 (m, 1H), 1.48 (m, 2H), 1.45 (s, 9H), 1.44 (s, 18H), 1.35 (m, 2H)

### Example 1-3. Preparation of Compound of Formula 6

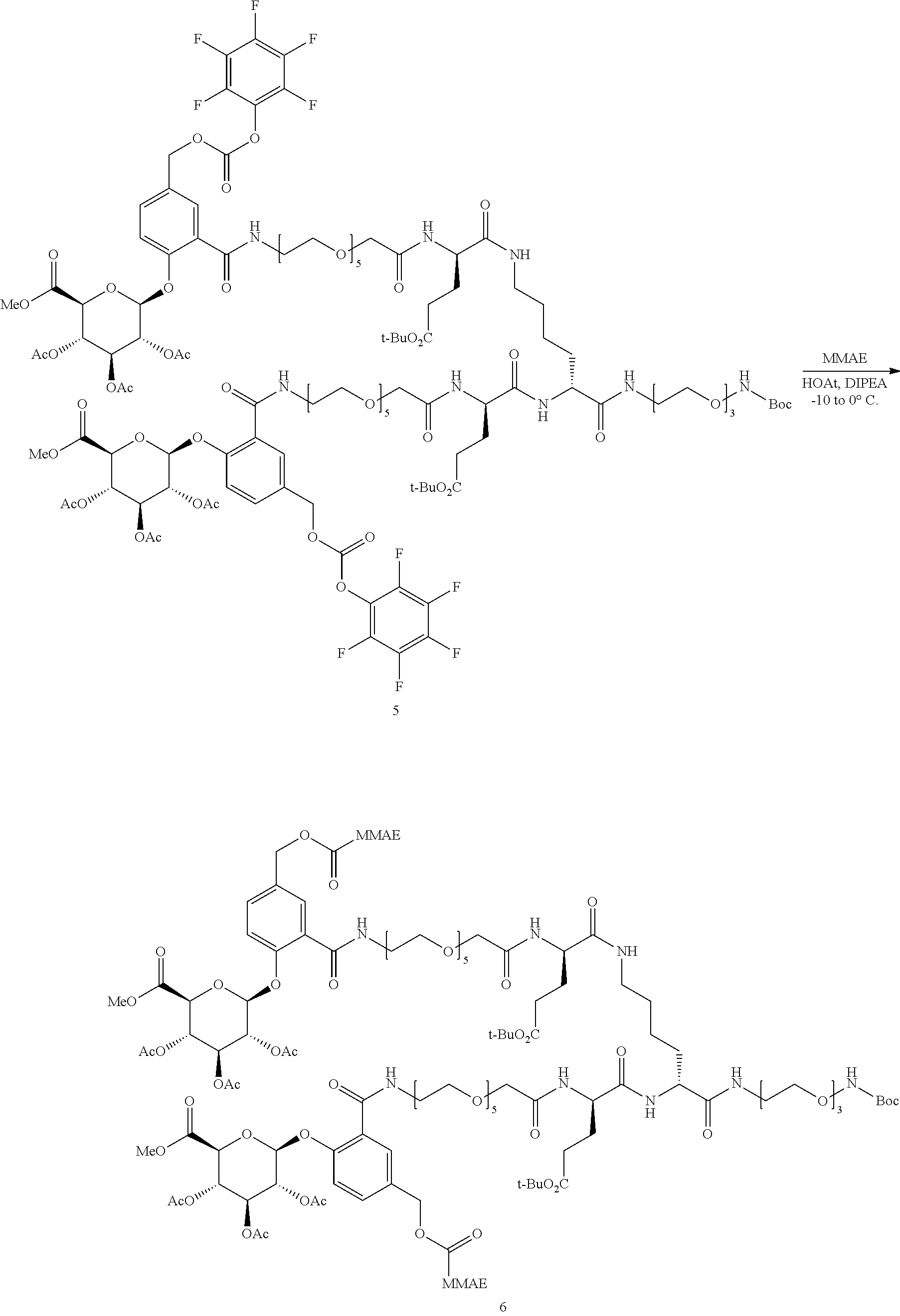

To a solution of the compound of Formula 5 (160 g, 56.3 mmol) dissolved in dimethylformamide (DMF) (1.6 L), monomethyl auristatin E (MMAE) (101 g, 141 mmol, 2.50 eq), diisopropylethylamine (DIEA) (36.4 g, 281 mmol, 5.00 eq), and hydroxyazabenzotriazole (HOAt) (7.67 g, 56.3 mmol, 1.00 eq) were sequentially added at −10° C.

The reaction mixture was stirred under a nitrogen atmosphere at 0° C. for 12 hours until the reaction was complete.

Subsequently, the reaction mixture, diluted with ethyl acetate (EtOAc) (3.60 L), was washed with 10% aqueous NH₄Cl solution (6.40 L), 5% aqueous NaHCO₃ solution (6.40 L), and brine (6.40 L), dried over anhydrous sodium sulfate, and concentrated under reduced pressure.

The concentrate was purified by column chromatography (SiO₂, 100% ethyl acetate→dichloromethane:methanol (10/1, v/v); TLC:SiO₂, dichloromethane:methanol (10/1, v/v), Rf=0.5) to obtain the compound of Formula 6 (200 g, 90.4%).

The Mass and NMR results for the compound of Formula 6 are as follows.

EI-MS m/z: [½M+H]⁺1870.3, [⅓M+H]⁺1247.1

¹H-NMR (400 MHz, MeOD), δ: 7.94 (m, 3H), 7.83 (m, 2H), 7.41-7.23 (m, 12H), 5.63 (m, 2H), 5.66-5.50 (m, 2H), 5.35 (m, 2H), 5.24-5.20 (m, 6H), 4.60-4.54 (m, 7H), 4.49-4.26 (m, 2H), 4.23 (m, 6H), 4.20-4.06 (br, 5H), 3.95 (m, 2H), 3.90 (m, 1H), 3.72-3.63 (m, 51H), 3.38-3.20 (m, 19H), 2.97-2.95 (m, 6H), 2.53-1.47 (m, 90H), 1.19 (m, 1H), 1.20-1.15 (m, 12H), 0.96-0.80 (m, 39H)

### *198 Example 1-4. Preparation of Compound of Formula 7

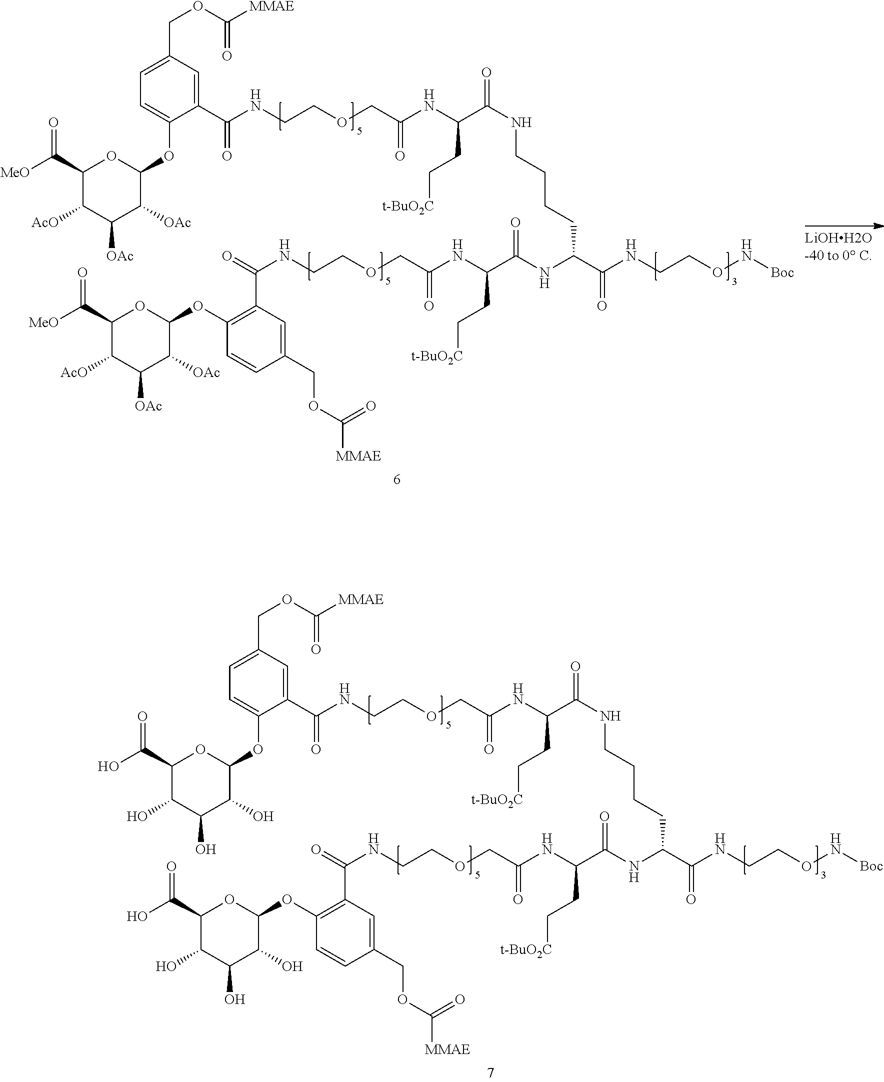

To a mixed solvent including methanol (MeOH) (2.85 L, 15 V) and tetrahydrofuran (THF) (2.85 L, 15 V) containing the dissolved compound of Formula 6 (200 g, 48.4 mmol), an aqueous solution of LiOH·H₂O (20.3 g, 484 mmol, 10.0 eq) dissolved in water (2.85 L, 15 V) was added dropwise over 30 minutes under a nitrogen atmosphere at a temperature of −40° C. to −30° C.

The reaction mixture was stirred under a nitrogen atmosphere at a temperature of −10 to 0° C. for 3.5 hours until the reaction was complete.

At the same temperature, the reaction mixture was neutralized with acetic acid (30.0 mL), concentrated, and then freeze-dried to obtain the unpurified compound of Formula 7 (190 g) as a white solid.

The Mass results for the compound of Formula 7 are as follows.

EI-MS m/z: [½M+H]⁺1730.1, [⅓M+H]⁺1153.7

### Example 1-5. Preparation of Compound of Formula 1

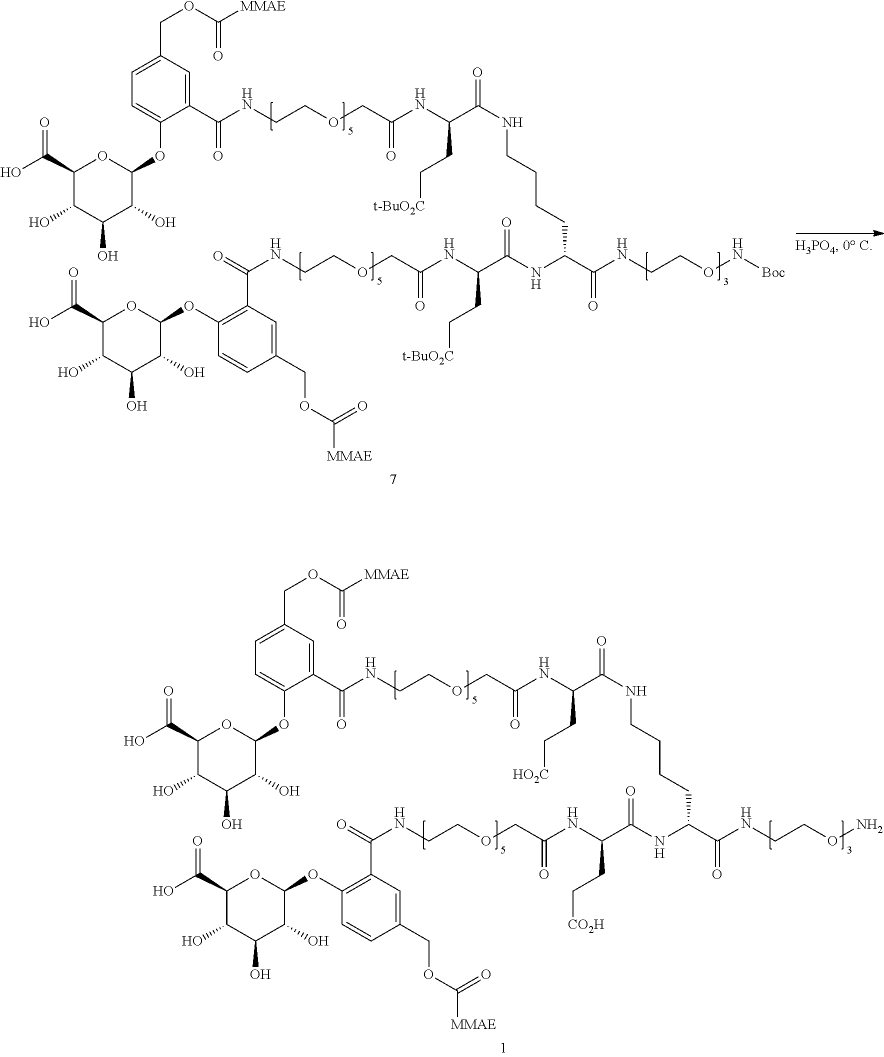

To a solution of the unpurified compound of Formula 7 (190 g) dissolved in dichloromethane dichloromethane (CH₂Cl₂, DCM) (3.8 L, 20 V), phosphoric acid (H₃PO₄) (380 mL, 2 V) was slowly added dropwise at a temperature of 0 to 5° C.

Subsequently, the reaction mixture was stirred at 0 to 5° C. for 2 hours until the reaction was complete.

The reaction mixture was dissolved in distilled water, purified by prep-HPLC (0.075% trifluoroacetic acid), and freeze-dried to obtain the compound of Formula 1 (76 g, 45% yield).

The Mass and NMR results for the compound of Formula 1 are as follows.

EI-MS m/z: [½M+H]⁺1623.2, [⅓M+H]⁺1082.6

¹H-NMR (400 MHz, MeOD), δ: 7.95 (m, 5H), 7.55 (m, 2H), 7.24-7.21 (m, 11H), 5.20-5.11 (m, 5H), 4.65-4.40 (m, 5H), 4.26-4.20 (m, 8H), 4.07-4.06 (br, 7H), 3.90-3.80 (m, 4H), 3.72-3.56 (m, 57H), 3.37-3.12 (m, 24H), 2.98-2.95 (m, 6H), 2.48-2.41 (m, 7H), 2.30-1.43 (m, 27H), 1.41-1.15 (m, 11H), 0.95-0.81 (m, 42H)

1. A method of preparing a linker-drug conjugate of Formula 1, the method comprising:
(1) obtaining a compound of Formula 4 by performing a condensation reaction between a compound of Formula 2 and a compound of Formula 3;
(2) obtaining a compound of Formula 5 by reacting the compound of Formula 4 and bis(perfluorophenyl) carbonate;
(3) obtaining a compound of Formula 6 by performing a condensation reaction between the compound of Formula 5 and monomethyl auristatin E (MMAE);
(4) obtaining a compound of Formula 7 by reacting the compound of Formula 6 and a base; and
(5) obtaining a compound of Formula 1 by reacting the compound of Formula 7 and an acid: 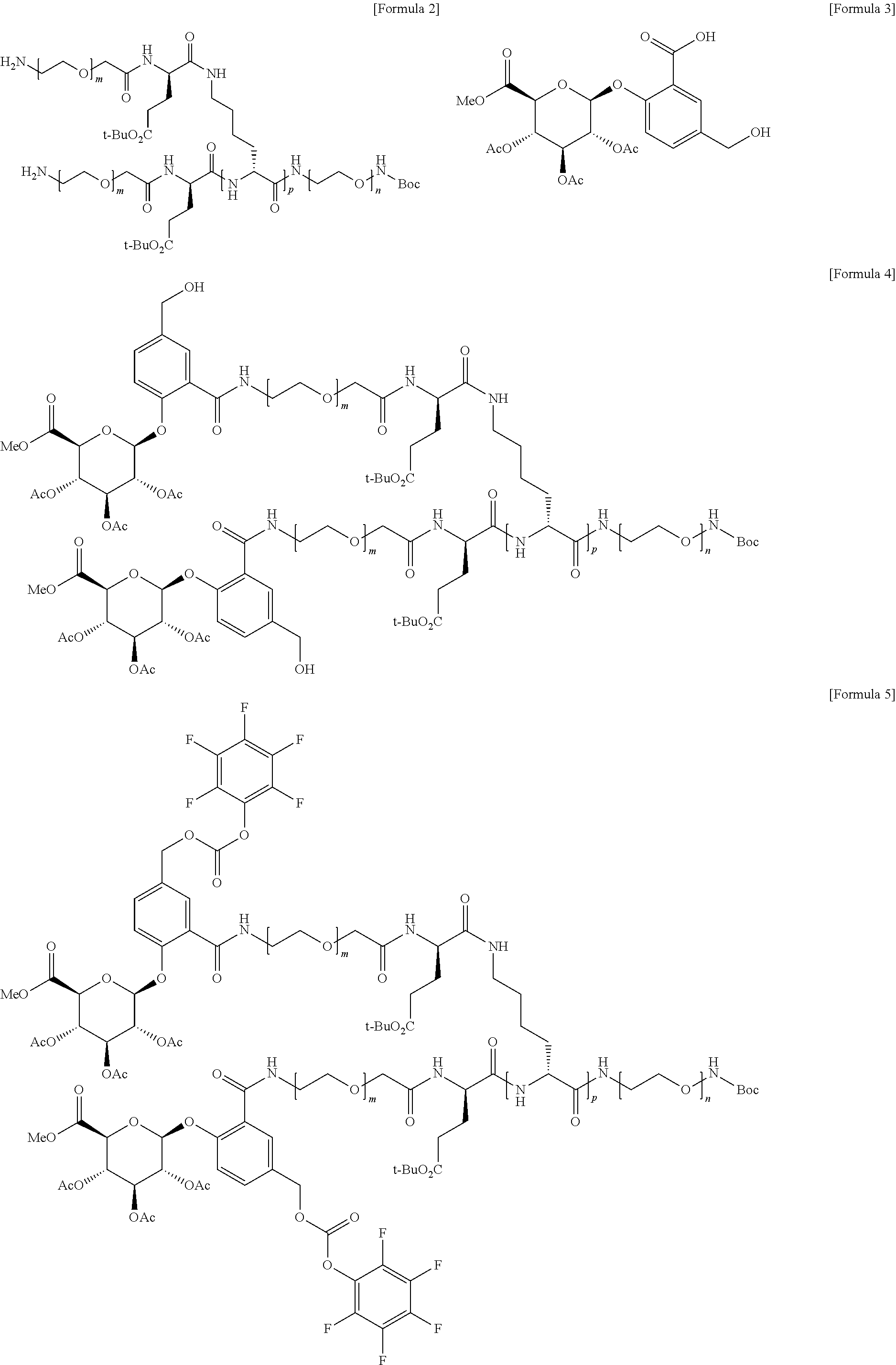 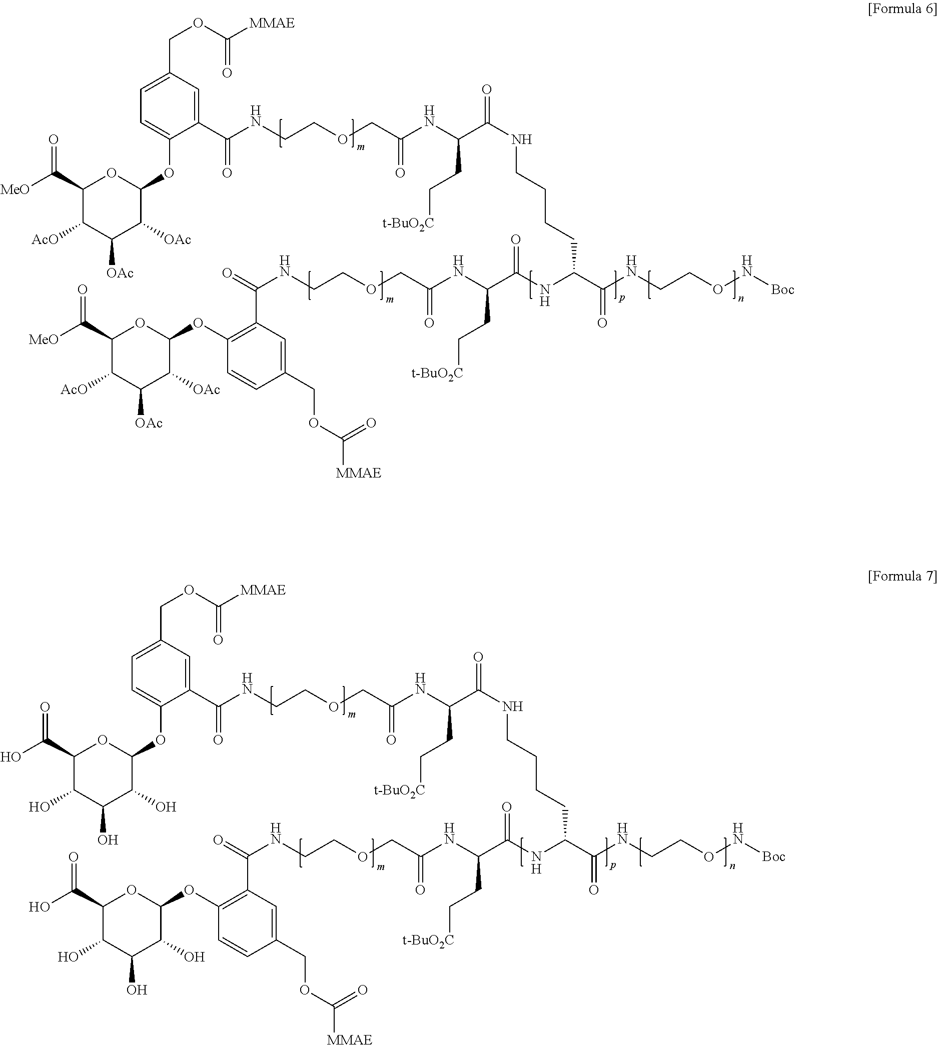 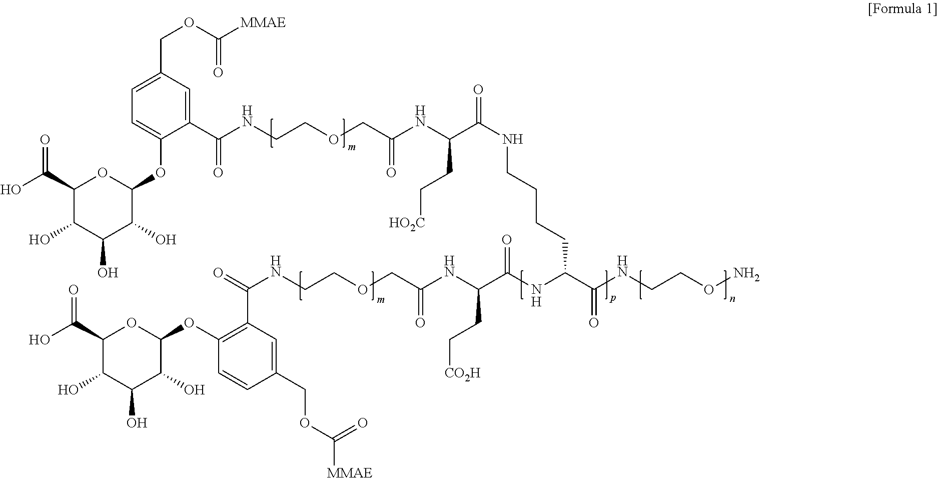
wherein, in Formulas 1 to 7,
p is an integer of 0 to 2,
m are the same or different from each other and are an integer of 1 to 6,
n is an integer of 1 to 6, and
MMAE is a compound of Formula 8.

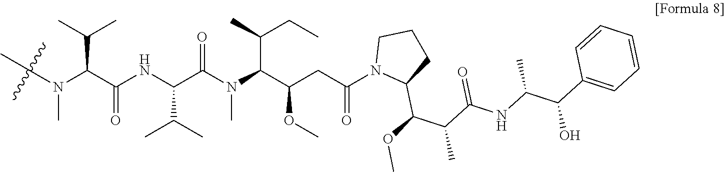 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

2. The method of claim 1,
wherein the condensation reaction of step (1) is performed in the presence of a condensation agent comprising one or more selected from the group consisting of 1-ethyl-3-(3-dimethylaminopropyl) carbodiimide hydrochloride, hydroxybenzotriazole, and N-methylmorpholine.

3. The method of claim 2,
wherein step (1) comprises:
(1-1) mixing the compound of Formula 2 and the compound of Formula 3, followed by cooling to −10 to 5° C.;
(1-2) adding the condensation agent at the aforementioned temperature; and
(1-3) stirring the mixture at room temperature.

4. The method of claim 3,
wherein the compound of Formula 2 and the compound of Formula 3 are mixed in an equivalent ratio of 1:1.5 to 1:3.

5. The method of claim 1,
wherein step (2) is performed in the presence of an organic base comprising one or more selected from the group consisting of pyridine, triethylamine, and N-methylmorpholine.

6. The method of claim 1,
wherein the condensation reaction of step (3) is performed in the presence of a condensation agent comprising one or more selected from the group consisting of hydroxyazabenzotriazole and hydroxybenzotriazole, and an organic base comprising one or more selected from the group consisting of diisopropylethylamine, triethylamine, and pyridine.

7. The method of claim 1,
wherein step (3) is adding the compound of Formula 5, monomethyl auristatin E (MMAE), the organic base, and the condensation agent at a temperature of 10 to 0° C., and reacting at a temperature of 0 to 25° C.

8. The method of claim 7,
wherein MMAE is included in an amount of 2 to 3 equivalents.

9. The method of claim 1,
wherein the base of step (4) comprises one or more selected from the group consisting of lithium hydroxide, sodium hydroxide, and calcium hydroxide.

10. The method of claim 1, further comprising, after step (4) and before step (5), a step of neutralizing the compound of Formula 7 to pH 6 to 8.

11. The method of claim 1,
wherein the acid of step (5) comprises one or more selected from the group consisting of phosphoric acid, trifluoroacetic acid, sulfuric acid, and acetic acid.

12. The method of claim 1,
wherein when p is 0, the linker-drug conjugate of Formula 1 is a compound of Formula 1-2: 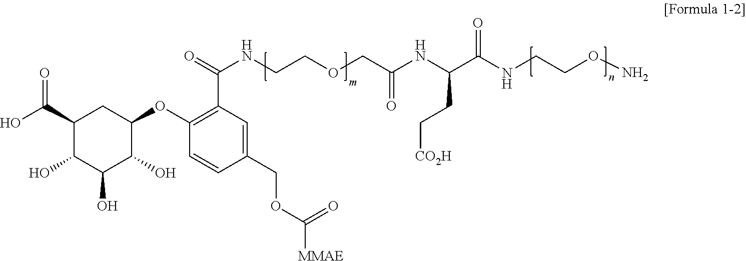
wherein m and n are as defined in Formula 1.

13. The method of claim 1,
wherein when p is 1, the linker-drug conjugate of Formula 1 is a compound of Formula 1-3: 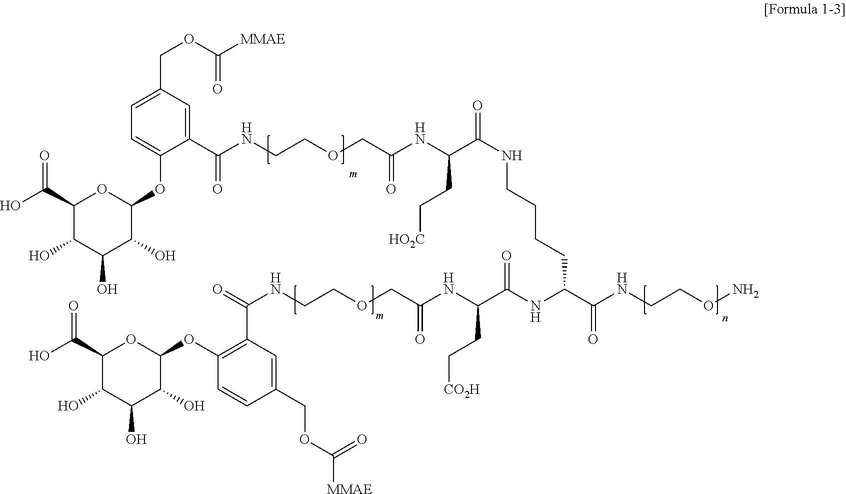
wherein m and n are as defined in Formula 1.

14. The method of claim 1,
wherein when p is 2, the linker-drug conjugate of Formula 1 is a compound of Formula 1-4: 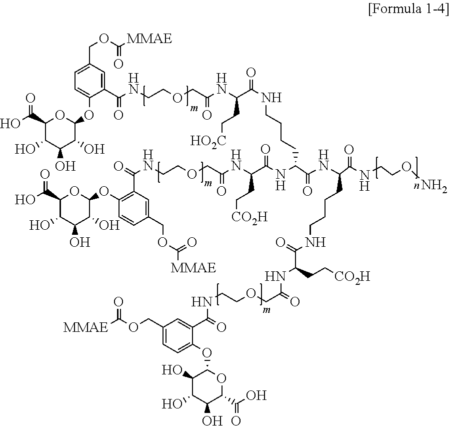
wherein m and n are as defined in Formula 1.
